(19) United States
(12) Patent Application Publication
(10) Pub. No.: US 2026/0325559 A1
(43) Pub. Date: Oct. 22, 2026

(54) Immune Activator Agent

(71) Applicant: Oryza Oil & Fat Chemical Co., Ltd., Ichinomiya-shi, Aichi (JP)

(72) Inventors: Kenchi MIYASAKA, Osaka (JP); Akari YONEDA, Toyama (JP); Shogo TAKEDA, Aichi (JP); Hiroshi SHIMODA, Aichi (JP); Hiromichi MURAI, Aichi (JP)

(73) Assignee: Oryza Oil & Fat Chemical Co., Ltd., Ichinomiya-shi, Aichi (JP)

(21) Appl. No.: 19/484,294

(22) PCT Filed: Dec. 11, 2024
(86) PCT No.: PCT/JP2024/043792

§ 371 Date: Nov. 13, 2025

(30) Foreign Application Priority Data
Dec. 22, 2023 (JP) 2023-216983
Apr. 5, 2024 (JP) 2024-061803

(51) Int. Cl. A61K 31/7028; A23L 33/10

(52) U.S. Cl. CPC A61K 31/7028; A23L 33/10

(57) ABSTRACT

The present invention provides a novel immune activator. The features of the invention for solving the above problem are as follows:
1. An IL-6 production enhancer for dendritic cells, comprising at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] as an active ingredient.
2. The IL-6 production enhancer according to item 1, wherein IL-6 production is induced by binding to TLR2 and/or TLR4.
3. A helper T-cell proliferation enhancer for dendritic cells, comprising at least one compound selected from the group consisting of GlcCer[d18:2(4E,8Z)/18:0] and GlcCer[d18:2(4E,8Z)/26:0] as an active ingredient.
4. An activator of antigen-presenting ability of dendritic cells, comprising GlcCer[d18:2(4E,8Z)/18:0] as an active ingredient.
5. An immune activator agent comprising, as an active ingredient, at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0].

### TECHNICAL FIELD

The present invention relates to an immune activator agent. The invention can be widely applied to pharmaceuticals, health foods, and general food products.

### BACKGROUND ART

When pathogens or bacteria enter a living body through a cut or mucous membrane, dendritic cells distributed throughout the body recognize and phagocytose the antigens, and the antigens are subsequently presented to leukocytes and lymphocytes. Thereafter, neutrophils, macrophages, and natural killer cells phagocytose and destroy the invading pathogens (innate immunity). In evaluating immune activation effects, tests are generally conducted using these cells as targets.

As defense mechanisms against foreign substances, there exist innate immunity and acquired immunity. In innate immune responses, for example, immune cells such as neutrophils, dendritic cells, and macrophages are known to produce cytokines in response to natural immune-activating substances derived from bacteria or viruses, thereby initiating subsequent immune reactions. The innate immune system is a defense mechanism commonly possessed by living organisms, and since it is generally nonspecific, it is characterized by a rapid response and effective function against a wide variety of infectious agents.

In dendritic cells and similar immune cells, innate immune responses are enhanced when pathogens or antigens bind to pattern recognition receptors (PRRs), leading to the activation of signaling pathways such as NF-κB and the subsequent release of various cytokines, including IL-6 and TNF-α. These PRRs include Toll-like receptors (TLRs) and C-type lectin receptors (CLRs) such as Dectin-1, DC-SIGN, and Mincle (Macrophage-Inducible C-type Lectin), each recognizing different types of antigens (see FIG. 2). Furthermore, dendritic cells and macrophages recognize antigens through PRRs and present these antigens to neutrophils or T cells (antigen presentation). When the recipient cells become activated and recognize the presented antigens, they phagocytose and eliminate pathogens, thereby activating the immune response. In addition, activation signaling among these cells after antigen presentation occurs via costimulatory molecules. For example, dendritic cells express CD40, CD80, CD86, and MHC class I and II molecules; CD80 and CD86 bind to CD28 on T cells, while CD40 binds to CD40L molecules on helper T cells, thereby mutually activating one another.

Dendritic cells possess phagocytic activity and also release IL-6. When dendritic cells mature, they exhibit superior antigen-presenting ability compared to other cells and release various cytokines. Among the cytokines released, interleukin-6 (IL-6) is produced in response to lipopolysaccharide (LPS) stimulation. Furthermore, mature dendritic cells have the ability to activate naïve T cells into effector T cells, thereby activating other immune cells as well. Examples include promoting the migration of neutrophils toward pathogens and enhancing the phagocytic activity of macrophages. Moreover, mature dendritic cells, through cytokines such as IL-6, induce the differentiation of naïve T cells into Th17 cells, which in turn promotes the migration of neutrophils.

### SUMMARY OF THE INVENTION

### Problem to be Solved by the Invention

In view of the foregoing background, the inventors of the present invention have discovered that certain novel sphingolipids possess an activity to promote interleukin-6 (IL-6) production in dendritic cells, thereby enhancing innate immunity, and have thus completed the present invention. Furthermore, tests conducted to determine the types of pattern recognition receptors (PRRs) to which these novel sphingolipids bind revealed that they bind to Toll-like receptors TLR2 and TLR4, thereby promoting antigen presentation, which also led to the completion of the invention. In addition, a mixed lymphocyte reaction (MLR) assay was carried out by coculturing dendritic cells and human peripheral blood mononuclear cells (PBMCs) to evaluate the proliferative capacity of CD4-positive helper T cells. The results demonstrated that these novel sphingolipids enhanced the proliferation of helper T cells and exhibited an activity to enhance the antigen-presenting capability of dendritic cells, thereby establishing the invention. Moreover, evaluation of cell surface markers (CD40, CD80, and CD86) involved in antigen presentation revealed that the novel sphingolipids increased the expression of CD40 and CD80 on dendritic cells. Accordingly, the objective of the present invention is to provide a novel immune activator agent capable of activating innate immunity.

### Means for Solving the Problems

The features of the present invention for solving the above problems are as follows:
1. An IL-6 production promoter for dendritic cells comprising, as an active ingredient, at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0].
2. The IL-6 production promoter according to item 1, wherein IL-6 production is mediated through binding to TLR2 and/or TLR4.
3. An agent for promoting helper T-cell proliferation by dendritic cells comprising, as an active ingredient, at least one compound selected from the group consisting of GlcCer[d18:2(4E,8Z)/18:0] and GlcCer[d18:2(4E,8Z)/26:0].
4. An agent for activating antigen-presenting function in dendritic cells comprising GlcCer[d18:2(4E,8Z)/18:0] as an active ingredient.
5. An immune activator agent comprising, as an active ingredient, at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0].
6. A food or beverage composition having an immune-activating effect, comprising, as an active ingredient, at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0].
7. A pharmaceutical composition having an immune-activating effect, comprising, as an active ingredient, at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0].
8. A method for activating human immunity, comprising: (a) isolating at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] from a rice bran extract; and (b) promoting IL-6 production in dendritic cells of a human by administering orally or parenterally an effective amount of the compound isolated in step (a) to the human.

### Effects of the Invention

The present invention provides that at least one type of ceramide selected from GlcCer[d18:2(4E,8Z)/18:0], GlcCer[d18:2(4E,8Z)/26:0], GlcCer[t18:1(8Z)/20:0], and Cer[t18:0/22:0] promotes interleukin-6 (IL-6) production in dendritic cells, thereby enhancing the migration of neutrophils toward pathogens and promoting the phagocytic activity of macrophages, which in turn strengthens the immune response. Furthermore, IL-6 production is mediated through binding to TLR2 and/or TLR4. Here, TLR2 recognizes lipoproteins that are components of peptidoglycan contained in the cell wall of Gram-positive bacteria. Accordingly, GlcCer[d18:2(4E,8Z)/18:0], GlcCer[d18:2(4E,8Z)/26:0], GlcCer[t18:1(8Z)/20:0], and Cer[t18:0/22:0] exhibit immune-activating effects against infections caused by Gram-positive bacteria. Examples of infections caused by Gram-positive bacteria include, but are not limited to, anthrax, diphtheria, enterococcal infections, Erysipelothrix infection, listeriosis, pneumococcal infections, Staphylococcus aureus infections, streptococcal infections, and toxic shock syndrome. TLR4, on the other hand, binds to lipopolysaccharides that constitute the outer surface of Gram-negative bacteria and similar pathogens. Accordingly, the compounds also exhibit immune-activating effects against infections caused by pathogens possessing lipopolysaccharides, such as Gram-negative bacteria. Examples of infections caused by Gram-negative bacteria include, but are not limited to, infections by Escherichia coli, Salmonella species, and Pseudomonas aeruginosa. Furthermore, GlcCer[d18:2(4E,8Z)/18:0] and GlcCer[d18:2(4E,8Z)/26:0] have an activity to activate helper T-cell proliferation mediated by dendritic cells, thereby exhibiting immune-activating effects. Moreover, GlcCer[d18:2(4E,8Z)/18:0] has an activity to activate the antigen-presenting function of dendritic cells.

### BRIEF DESCRIPTION OF THE DRAWINGS

FIG. 1 is a graph showing the results of evaluating the effect on IL-6 production activity in iMylc® cells (immature myeloid cells induced by differentiation from iPS cells).

FIG. 2 is a diagram showing pattern recognition receptors present on dendritic cells.

FIG. 3 is a graph showing the effects of various TLR and CLR (Dectin-1, DC-SIGN, Mincle) inhibitors on the IL-6 production activity of GlcCer[d18:2(4E,8Z)/18:0].

FIG. 4 is a diagram showing the gating strategy used for the mixed lymphocyte reaction (MLR) using αMylc® cells and human PBMCs.

FIG. 5 is a graph and diagram showing the effects of GlcCer 1-4, which exhibited IL-6 production activity, on the cell proliferative capacity in an MLR using αMylc® cells and CD4-positive PBMCs.

FIG. 6 is a graph showing the effects of GlcCer 1-4 on the expression of CD80, CD86, and CD40 on αMylc® cells.

FIG. 7 is a diagram showing the effects of GlcCer 1-4 on the expression of CD80 and CD40 on αMylc® cells (pseudocolor image, one representative example).

### MODE FOR CARRYING OUT THE INVENTION

The immune activator agent of the present invention is characterized by comprising, as an active ingredient, at least one type of ceramide selected from GlcCer[d18:2(4E,8Z)/18:0], GlcCer[d18:2(4E,8Z)/26:0], GlcCer[t18:1(8Z)/20:0], and Cer[t18:0/22:0].

Each of the above ceramides is represented by the following Chemical Formulae 1-4.

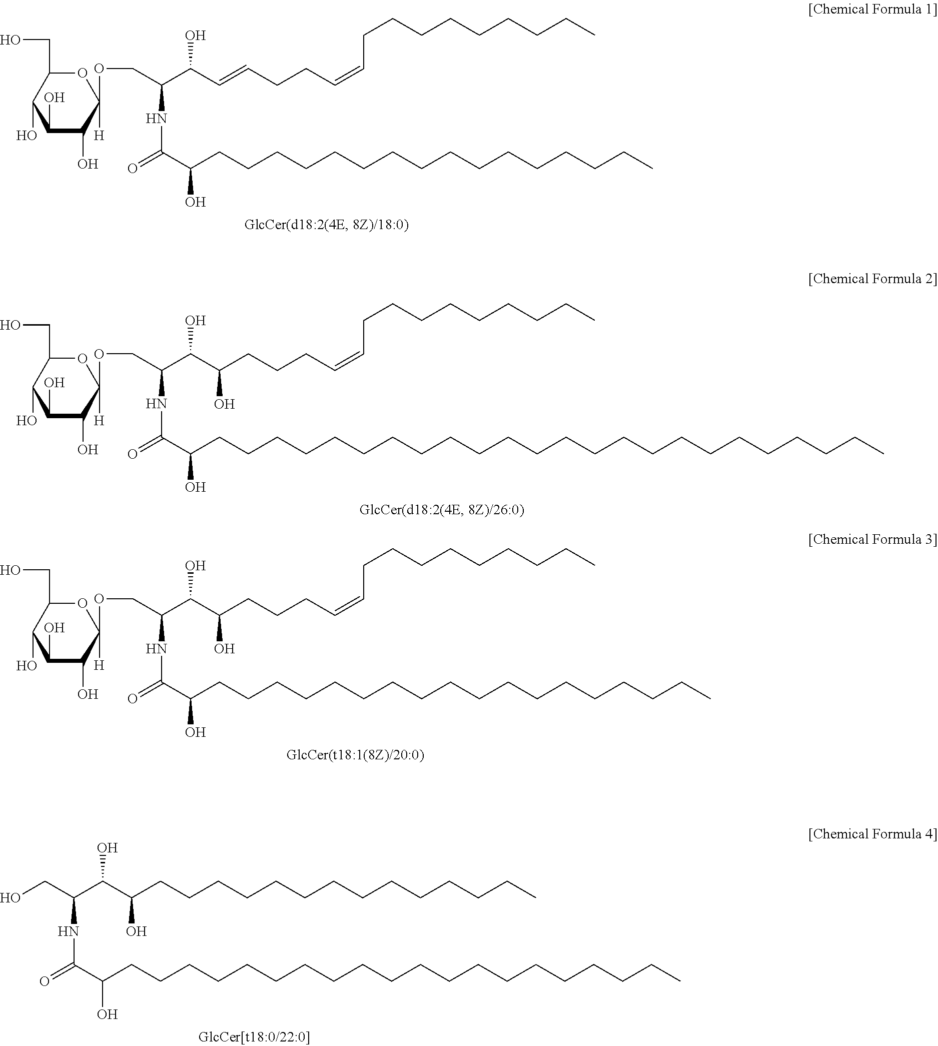

The method for producing the above ceramides is not particularly limited, but they can be obtained by extracting rice bran with a polar solvent to obtain a rice bran extract, and then isolating the ceramides from the resulting extract.

The term “rice bran extract” mentioned above is not particularly limited as long as it is a material produced in the process of manufacturing rice bran oil. For example, the rice bran extract may be obtained by extracting rice bran with an organic solvent and then precipitating the resulting extract, although the method is not limited thereto. In addition, the rice bran extract may also be one obtained by extracting a by-product generated during the manufacturing process of rice bran oil.

The method for isolating the above ceramides from the rice bran extract is not particularly limited. For example, the ceramides can be isolated by chromatography using activated clay, activated carbon, silica gel, alumina, diatomaceous earth, synthetic adsorbents, or ion exchange resins, or by removing components other than the ceramides through adsorption, decomposition, precipitation, filtration, dissolution, or distillation. Alternatively, commercially available rice-derived ceramide extracts may be used, such as “Oryza Ceramide®” manufactured by Oryza Oil & Chemical Co., Ltd.

The immune activator agent of the present invention can be used as a material for various food and beverage products. Examples of such foods and beverages include general food products such as confectionery (e.g., gum, candy, caramel, chocolate, cookies, snacks, jelly, gummies, and tablets), noodles (e.g., soba, udon, ramen), dairy products (e.g., milk, ice cream, yogurt), seasonings (e.g., miso, soy sauce), soups, and beverages (e.g., juice, coffee, tea, carbonated drinks, and sports drinks), as well as health foods (e.g., tablets, capsules) and nutritional supplements (e.g., energy drinks). The immune activator agent of the present invention may be appropriately blended into these food and beverage products.

Various components may be added to these food and beverage products depending on their type. For example, food materials such as glucose, fructose, sucrose, maltose, sorbitol, stevioside, corn syrup, lactose, citric acid, tartaric acid, malic acid, succinic acid, lactic acid, L-ascorbic acid, dl-α-tocopherol, sodium erythorbate, glycerin, propylene glycol, glycerin fatty acid esters, polyglycerin fatty acid esters, sucrose fatty acid esters, sorbitan fatty acid esters, propylene glycol fatty acid esters, arabic gum, carrageenan, casein, gelatin, pectin, agar, vitamin B group, nicotinamide, calcium pantothenate, amino acids, calcium salts, coloring agents, flavoring agents, and preservatives may be used.

As a specific method of preparation, the immune activator agent of the present invention may be spray-dried or freeze-dried together with powdered cellulose, and the resulting material may be formed into a powder, granules, tablets, or a solution, thereby allowing the agent to be easily incorporated into food and beverage products such as instant foods. Alternatively, the agent of the present invention may be dissolved in fats and oils, ethanol, glycerin, or mixtures thereof to prepare a liquid form, which can then be added to beverages or solid foods. If necessary, the agent may also be mixed with a binder such as arabic gum or dextrin to form a powder or granules, which can be added to beverages or solid foods.

When the agent of the present invention is applied to food and beverage products, the amount to be added is preferably such that the total content of the active ingredient in the food or beverage is 1 to 20 wt % or less, since the primary purpose is disease prevention and health maintenance.

The immune activator agent of the present invention may also be used as a material for pharmaceuticals (including medicines and quasi-drugs). The agent of the present invention can be suitably blended with pharmaceutical raw materials to produce pharmaceutical preparations. Examples of pharmaceutical raw materials that can be incorporated into the agent of the present invention include, for example: excipients, such as glucose, lactose, sucrose, sodium chloride, starch, calcium carbonate, kaolin, crystalline cellulose, cacao butter, hardened vegetable oils, and talc; binders, such as distilled water, physiological saline, aqueous ethanol, simple syrup, glucose solution, starch paste, gelatin solution, carboxymethyl cellulose, potassium phosphate, and polyvinylpyrrolidone; disintegrants, such as sodium alginate, agar, sodium bicarbonate, calcium carbonate, sodium lauryl sulfate, monoglyceride stearate, starch, lactose, powdered arabic gum, gelatin, and ethanol; disintegration inhibitors, such as sucrose, stearin, cacao butter, and hydrogenated oils; absorption promoters, such as quaternary ammonium bases and sodium lauryl sulfate; adsorbents, such as glycerin, starch, lactose, kaolin, bentonite, and silica; and lubricants, such as purified talc, stearic acid salts, and polyethylene glycol.

The immune activator agent of the present invention can generally be administered orally in the form of tablets, pills, soft or hard capsules, fine granules, powders, granules, or liquids. However, parenteral administration may also be employed. When administered as a parenteral preparation, the agent may be administered in the form of a solution, or in a state in which dispersing agents, suspending agents, or stabilizers are added, by local tissue injection, intradermal, subcutaneous, intramuscular, or intravenous injection. The agent may also be administered in the form of suppositories, or as an ophthalmic preparation.

The dosage may vary depending on the method of administration, the condition of the disease, the age of the patient, and other factors. In adults, the effective amount of the active ingredient is usually 0.5 to 5000 mg per day, while in children, it is generally about 0.5 to 3000 mg per day. The blending ratio of the immune activator agent may be appropriately adjusted depending on the dosage form. Typically, when administered orally or through mucosal absorption, the concentration of the active ingredient is about 0.3 to 15.0 wt %, and when administered parenterally, it is preferably about 0.01 to 10 wt %. It should be noted that the actual dosage may vary depending on various conditions, and in some cases, a smaller amount than the above range may be sufficient, while in other cases, an amount exceeding the above range may be required.

### EXAMPLES

The following examples are provided to illustrate embodiments of the present invention. It should be understood that these examples are presented solely for confirming the various actions and effects of the immune activator agent obtained according to the present invention, and that the scope of the present invention is not limited to the products or production methods described herein.

### Example: Preparation of Ceramides

### (1) Preparation of Glucosylceramides

Oryza Ceramide® (manufactured by Oryza Oil & Chemical Co., Ltd.) was subjected to rough fractionation using medium-pressure preparative liquid chromatography. Fractionation was performed using a Universal Silica Gel Column Premium (manufactured by Yamazen Corporation) under the following elution conditions: hexane:ethyl acetate (9:1→8:2→7:3→5:5)→ethyl acetate→chloroform:methanol (9:1→8:2→7:3→5:5)→methanol. The chloroform:methanol (8:2) fraction was then subjected to reversed-phase HPLC (COSMOSIL C18 MS-II, methanol) to purify four types of glucosylceramides. Each purified glucosylceramide was identified as the glucosylceramides represented by Chemical Formulae 1, 2, 3, and 5 by comparing their NMR and MS spectra with those described in the literature values [1]-[8] below.
1. Inagaki et al., Chem. Pharm. Bull., 52(11), 1307-1311 (2004)
2. Jung J. H. et al., J. Nat. Prod., 59, 319-322 (1996)
3. Liu H. et al., Phytochemistry, 49(8), 2403-2408 (1998)
4. Ryu J. et al., Arch. Pharm. Res., 26(2), 138-142 (2003)
5. Pittaya T. et al., Chem. Pharm. Bull., 52(1), 27-32 (2004)
6. Kang S. S. et al., Chem. Pharm. Bull., 49, 321-323 (2001)
7. Luo Y. et al., Lipid, 39(9), 907-914 (2004)
8. Zhang W. K. et al., Chem. Phys. Lipids, 148, 77-83 (2007)

The compound represented by Chemical Formula 5 is as follows.

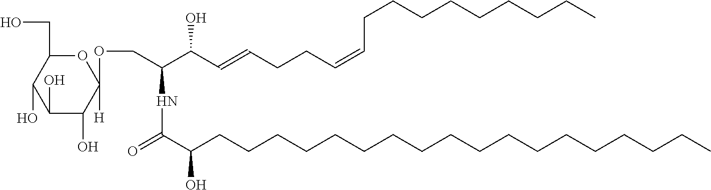

### (2) Preparation of the Compound Represented by Chemical Formula (4) (Cer[t18:0/22:0])

An ethanol extract of rice bran was used as the starting material. The extract (10 g) was adsorbed onto silica gel (22 g) to prepare a “mabushi” mixture (adsorbed sample). The mixture was then subjected to medium-pressure flash chromatography (manufactured by Yamazen Corporation) for fractionation. Specifically, the “mabushi” sample was loaded onto a medium-pressure silica gel flash column (Universal Column Silica Gel, 3 L), and sequentially fractionated using the following solvent systems: hexane:ethyl acetate, and chloroform:methanol. Detection was performed using an evaporative light scattering detector (ELSD).

TABLE 1
Conditions for Medium Pressure Flash Chromatography Separation

| Time (min) | Hexane (%) | Ethyl acetate (%) |
|---|---|---|
| 0 | 90 | 10 |
| 5 | 90 | 10 |
| 10 | 80 | 20 |
| 15 | 80 | 20 |
| 20 | 70 | 30 |
| 25 | 70 | 30 |
| 30 | 50 | 50 |
| 40 | 50 | 50 |
| 45 | 0 | 100 |
| 55 | 0 | 100 |

| Time (min) | Chloroform (%) | Methanol (%) |
|---|---|---|
| 0 | 90 | 10 |
| 5 | 90 | 10 |
| 10 | 80 | 20 |
| 15 | 80 | 20 |
| 20 | 70 | 30 |
| 25 | 70 | 30 |
| 30 | 50 | 50 |
| 40 | 50 | 50 |
| 45 | 0 | 100 |
| 50 | 0 | 100 |

Among the fractions eluted with chloroform:methanol (90:10→80:20) for 5-10 minutes, fractions 17 and 18 were collected and concentrated to obtain a crude fraction. This crude fraction was repeatedly purified by preparative HPLC (C18 column; CAPCELL PAK C18, 20×250 mm, manufactured by Osaka Soda Co., Ltd.) using tetrahydrofuran:methanol (1:9) as the mobile phase. The proton and carbon NMR spectra and mass spectrum of the resulting compound were compared with the literature values (Gao J. M. et al., Chem. Phys. Lipids, 131, 205-213 (2004)), confirming that the compound was Cer[t18:0/22:0] represented by Chemical Formula 4.

### Test Example 1: Evaluation of IL-6 Production Activity in Immature Dendritic Cells

### (1) Test Method

In this experiment, iMylc® cells (immature myeloid cells induced by differentiation from iPS cells) purchased from Mycen Technologies Inc. were used. These cells were cultured and subjected to evaluation tests using the dedicated maintenance medium for iMylc® cells provided by Mycen Technologies Inc.

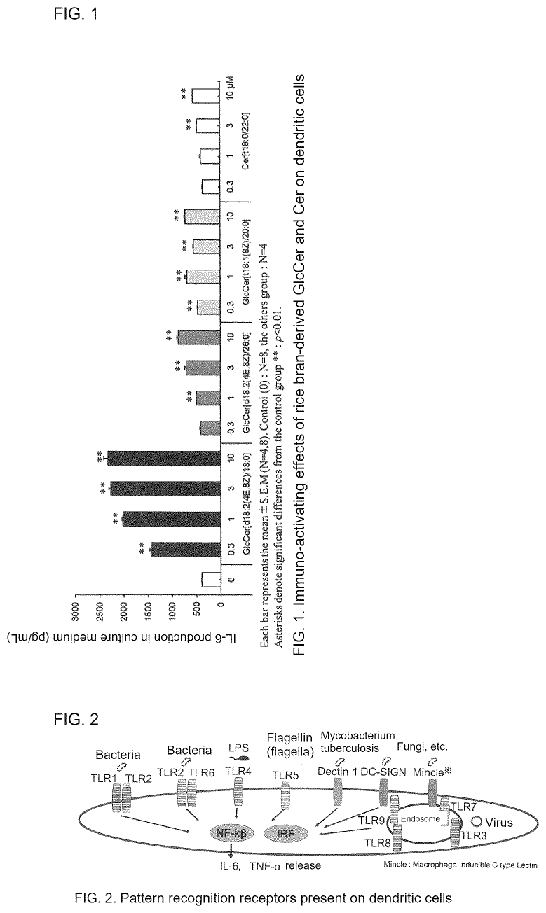

The iMylc® cells were seeded into 96-well plates (1.0×10⁴ cells/well/100 μL). After adding 100 μL/well of medium containing the test substance, the cells were incubated for 20 hours at 37 °C under 5% CO₂. The final concentration of DMSO was adjusted to 0.1% (v/v). After incubation, the amount of IL-6 produced in the culture supernatant was measured using a commercially available kit (Human IL-6 ELISA MAX™ Deluxe Set, BioLegend) in accordance with the manufacturer's instructions. The results are shown in FIG. 1.

### (2) Results and Effects of the Example in Test Example 1

As a result of evaluating the immune-activating effects of various GlcCer and Cer compounds on dendritic cells, a marked increase in IL-6 production was observed for GlcCer[d18:2(4E,8Z)/18:0]. In addition, when compared in terms of chemical structure, GlcCer[d18:2(4E,8Z)/26:0], which has a fatty acid chain length of 26 carbons, exhibited significant activity. Furthermore, GlcCer[t18:1(8Z)/20:0], which has a different sphingoid base structure, and Cer[t18:0/22:0], which lacks a sugar moiety, also showed significant activity.

Accordingly, GlcCer[d18:2(4E,8Z)/18:0], GlcCer[d18:2(4E,8Z)/26:0], GlcCer[t18:1(8Z)/20:0], and Cer[t18:0/22:0] were confirmed to have IL-6 production-promoting activity in dendritic cells. This activity enhances neutrophil migration toward pathogens and promotes macrophage phagocytic activity, thereby strengthening immune responses. These findings demonstrate that the compounds are useful as immune activator agents.

### Test Example 2: Evaluation of Pattern Recognition Receptor (PRR) Binding Activity in Dendritic Cells

### (1) Test Method

The test was conducted using iMylc® cells with various TLR and CLR inhibitors.

The iMylc® cells (immature myeloid cells derived from iPS cells) were cultured and subjected to evaluation using the dedicated maintenance medium for iMylc® cells provided by Mycen Technologies Inc.

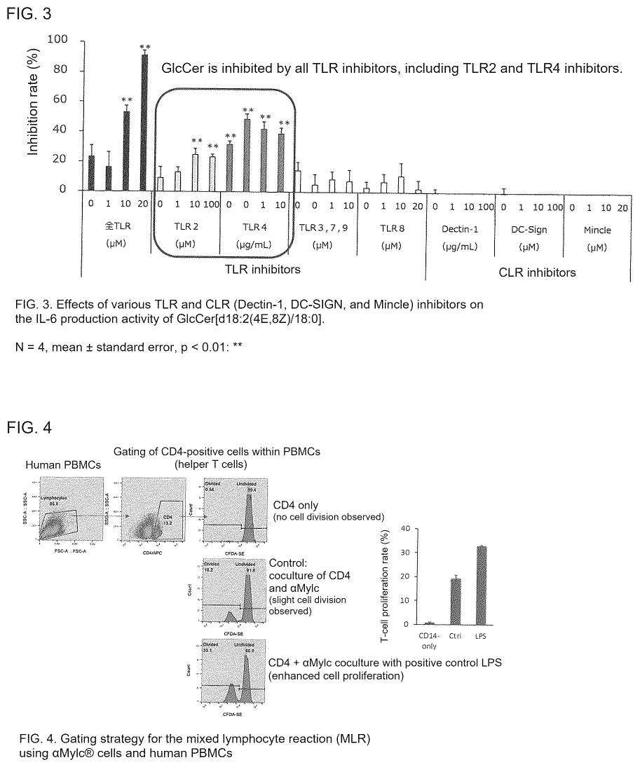

The iMylc® cells were seeded into 96-well plates (1.0×10⁴ cells/well/100 μL). After adding 50 μL/well of medium containing the respective inhibitors (listed below), the cells were incubated for 4 hours at 37 °C under 5% CO₂. Subsequently, 50 μL/well of medium containing GlcCer[d18:2(4E,8Z)/18:0] was added, and the cells were further incubated for 20 hours at 37 °C under 5% CO₂. The final concentration of DMSO was adjusted to 0.1% (v/v). After incubation, the amount of IL-6 produced in the culture supernatant was measured (N=4) using a commercially available kit (Human IL-6 ELISA MAX™ Deluxe Set, BioLegend) in accordance with the manufacturer's instructions. The results are shown in FIG. 3.

### <List of Inhibitors>

TLR inhibitors: Chloroquine diphosphate (FUJIFILM Wako Pure Chemical Corporation)
TLR2 inhibitor: MMG-11 (MedChemExpress)
TLR4 inhibitor: LPS-RS (InvivoGen)
TLR3, 7, and 9 inhibitor: ODN2088 (InvivoGen)
TLR8 inhibitor: CU-CPT9a (InvivoGen)
Dectin-1 inhibitor: Laminarin (Sigma-Aldrich)
DC-SIGN inhibitor: Fucosyllactose Laminarin (Sigma-Aldrich)
Mincle expression inhibitor: Isoliquiritigenin (Tokyo Chemical Industry Co., Ltd.)

It should be noted that each inhibitor was used at a concentration that showed no cytotoxicity toward iMylc® cells.

### (2) Results and Effects of the Example in Test Example 2

As a result, IL-6 production induced by GlcCer[d18:2(4E,8Z)/18:0] was inhibited by all TLR inhibitors in a concentration-dependent manner, and at a high concentration (20 μM), more than 90% inhibition was observed. In particular, the inhibition was most pronounced with the TLR2 and TLR4 inhibitors.

These results indicate that GlcCer[d18:2(4E,8Z)/18:0] binds to TLR2 and TLR4 on dendritic cells and promotes IL-6 production (immune activation). (FIG. 3) It is also considered that GlcCer[d18:2(4E,8Z)/26:0], GlcCer[t18:1(8Z)/20:0], and Cer[t18:0/22:0] promote IL-6 production (i.e., immune activation) through the same mechanism.

### Test Example 3: Evaluation of Helper T Cell Proliferation Induced by Dendritic Cells

### (1) Test Method

The test was conducted using a mixed lymphocyte reaction (MLR). In this method, αMylc® cells (derived from human peripheral blood) that had shown IL-6 production-promoting activity were treated with GlcCer[d18:2(4E,8Z)/18:0], GlcCer[t18:1(8Z)/20:0], GlcCer[d18:2(4E,8Z)/20:0], or GlcCer[d18:2(4E,8Z)/26:0] at concentrations of 1 or 10 μM for 24 hours.

During this period, peripheral blood mononuclear cells (PBMCs) isolated from human blood were labeled with the cell membrane-permeable dye carboxyfluorescein diacetate succinimidyl ester (CFDA-SE) by incubation for 7 minutes. After staining, αMylc® cells and PBMCs were mixed at a 1:2 ratio (2×10⁴ cells: 4×10⁴ cells) and cocultured for 7 days.

Subsequently, the mean fluorescence intensity (MFI) of CD4 (APC-stained; helper T cell marker) and CFDA (cell proliferation indicator) was quantified by flow cytometry (N=3). In the present MLR assay, the activation index was defined as the proliferation rate of helper T cells, and the gating strategy used in this analysis is shown in FIG. 4.

### (2) Results and Effects of the Example in Test Example 3

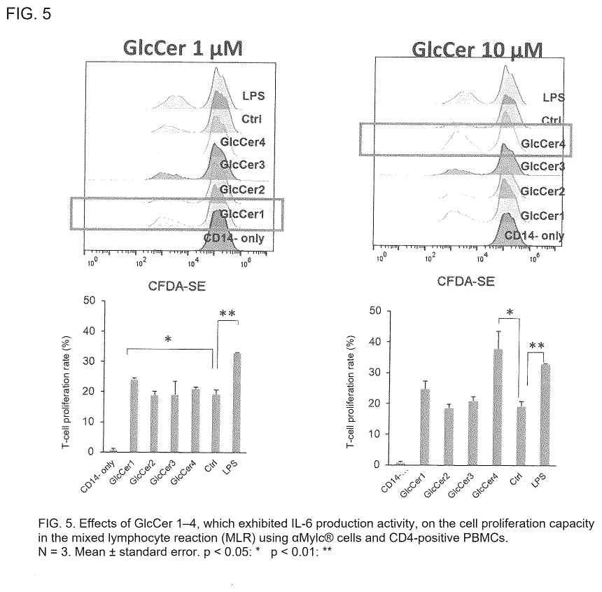

The results of Test Example 3 are shown in FIG. 5.

As shown in FIG. 5, a significant increase in T-cell proliferation rate was observed with 1 μM GlcCer[d18:2(4E,8Z)/18:0] and 10 μM GlcCer[d18:2(4E,8Z)/26:0].

These results clearly indicate that the above GlcCer compounds enhance the antigen-presenting ability of dendritic cells.

### Test Example 4: Evaluation of Antigen-Presenting Activity of Dendritic Cells

### (1) Test Method

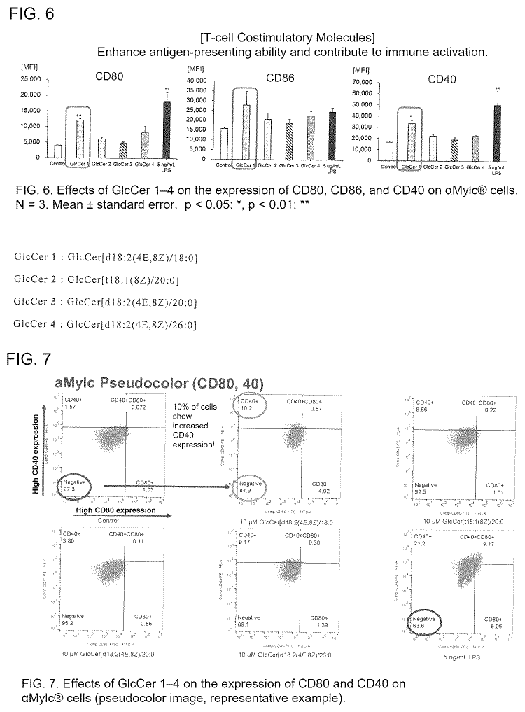

αMylc® cells (derived from human peripheral blood) were used in this experiment. The αMylc® cells were seeded into 6-well plates (1.0×10⁵ cells/well/1000 μL). After adding 1000 μL/well of medium containing GlcCer[d18:2(4E,8Z)/18:0], GlcCer[t18:1(8Z)/20:0], GlcCer[d18:2(4E,8Z)/20:0], or GlcCer[d18:2(4E,8Z)/26:0], the cells were incubated for 24 hours at 37 °C under 5% CO₂. The final concentration of DMSO was adjusted to 0.1% (v/v). After incubation, the cells were stained with the antibodies listed below according to the flow cytometry procedure, and the mean fluorescence intensity (MFI) of each cell-surface marker was measured using a flow cytometer (N=3). The results are shown in FIG. 6, and the pseudocolor images for CD40 and CD86 are shown in FIG. 7.

### <Antibodies>

PE-CD40 (BioLegend)
FITC-CD80 (BioLegend)
FITC-CD86 (BioLegend)

### (2) Results and Effects of the Example in Test Example 4

As shown in FIG. 6, a significant increase in the expression of CD80 and CD40 was observed in response to GlcCer[d18:2(4E,8Z)/18:0], while CD86 showed a tendency to increase. Accordingly, FIG. 7 presents the pseudocolor results for CD40 and CD86, in which the plots for GlcCer[d18:2(4E,8Z)/18:0] were overall shifted upward to the right compared with the control.

These findings confirm that GlcCer[d18:2(4E,8Z)/18:0] possesses an activity to activate the antigen-presenting ability of dendritic cells.

### Formulation Example 1: Chewing Gum

The following examples illustrate formulations containing the agent (ceramide compounds) according to the present invention. However, these examples are not intended to limit the scope of the invention.

| Ingredient | Content (wt %) |
|---|---|
| Sugar | 53.45 |
| Gum base | 20.0 |
| Glucose | 10.0 |
| Starch syrup | 16.0 |
| Flavor | 0.5 |
| Ceramide compounds | 0.05 |
| Total | 100.0 wt % |

### Formulation Example 2: Gummy Candy

| Ingredient | Content (wt %) |
|---|---|
| Reduced starch syrup | 40.9 |
| Granulated sugar | 20.0 |
| Glucose | 20.0 |
| Gelatin | 4.7 |
| Water | 9.68 |
| Yuzu (Japanese citrus) juice | 4.0 |
| Yuzu (Japanese citrus) flavor | 0.6 |
| Coloring agent | 0.02 |
| Ceramide compounds | 0.1 |
| Total | 100.0 wt % |

### Formulation Example 3: Candy

| Ingredient | Content (wt %) |
|---|---|
| Sugar | 50.36 |
| Starch syrup | 33.0 |
| Water | 14.4 |
| Organic acid | 2.0 |
| Flavor | 0.2 |
| Ceramide compounds | 0.04 |
| Total | 100.0 wt % |

### Formulation Example 4: Yogurt (Hard or Soft Type)

| Ingredient | Content (wt %) |
|---|---|
| Milk | 41.5 |
| Skim milk powder | 5.8 |
| Sugar | 8.0 |
| Agar | 0.15 |
| Gelatin | 0.1 |
| Lactic acid bacteria | 0.005 |
| Ceramide compounds | 0.04 |
| Flavor | trace amount |
| Water | balance |
| Total | 100.0 wt % |

### Formulation Example 5: Soft Drink

| Ingredient | Content (wt %) |
|---|---|
| High-fructose corn syrup | 30.0 |
| Emulsifier | 0.5 |
| Ceramide compounds | 0.03 |
| Flavor | trace amount |
| Purified water | balance |
| Total | 100.0 wt % |

### Formulation Example 6: Compressed Candy

| Ingredient | Content (wt %) |
|---|---|
| Sugar | 76.4 |
| Glucose | 19.0 |
| Sucrose fatty acid ester | 0.2 |
| Ceramide compounds | 0.05 |
| Purified water | 4.35 |
| Total | 100.0 wt % |

### Formulation Example 7: Soft Capsule

| Ingredient | Content (wt %) |
|---|---|
| Brown rice germ oil | 47.9 |
| Yuzu (Japanese citrus) seed oil | 40.0 |
| Emulsifier | 12.0 |
| Ceramide compounds | 0.1 |
| Total | 100.0 wt % |

### Formulation Example 8: Tablet

| Ingredient | Content (wt %) |
|---|---|
| Lactose | 54.9 |
| Crystalline cellulose | 30.0 |
| Starch hydrolysate | 10.0 |
| Glycerin fatty acid ester | 5.0 |
| Ceramide compounds | 0.1 |
| Total | 100.0 wt % |

### INDUSTRIAL APPLICABILITY

As described above, the present invention provides an immune activator agent comprising a novel component as an active ingredient.

What is claimed is:

1. An IL-6 production enhancer for dendritic cells, comprising at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] as an active ingredient.

2. The IL-6 production enhancer according to claim 1, wherein IL-6 production is induced by binding to TLR2 and/or TLR4.

3. A helper T-cell proliferation enhancer for dendritic cells, comprising at least one compound selected from the group consisting of GlcCer[d18:2(4E,8Z)/18:0] and GlcCer[d18:2(4E,8Z)/26:0] as an active ingredient.

4. An activator of antigen-presenting ability of dendritic cells, comprising GlcCer[d18:2(4E,8Z)/18:0] as an active ingredient.

5. An immune activator agent comprising at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] as an active ingredient.

6. A food or beverage composition having an immune-activating effect, comprising at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] as an active ingredient.

7. A pharmaceutical composition (including medicines and quasi-drugs) having an immune-activating effect, comprising at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] as an active ingredient.

8. A method for activating human immunity, comprising:
(a) isolating at least one compound selected from the group consisting of GlcCer[d18:2(4E,8Z)/18:0], GlcCer[d18:2(4E,8Z)/26:0], GlcCer[t18:1(8Z)/20:0], and Cer[t18:0/22:0] from a rice bran extract; and
(b) administering an effective amount of the compound isolated in step (a) to a human by oral or parenteral administration, thereby promoting IL-6 production in dendritic cells of the human.

9. A method for activating human immunity, comprising:
(a) isolating four compounds, GlcCer[d18:2(4E,8Z)/18:0], GlcCer[d18:2(4E,8Z)/26:0], GlcCer[t18:1(8Z)/20:0], and Cer[t18:0/22:0], from a rice bran extract; and
(b) promoting IL-6 production in dendritic cells of a human by administering an effective amount of the four compounds isolated in step (a) to the human via oral administration or parenteral administration.

10. A method for activating human immunity, comprising:
(a) isolating Cer[t18:0/22:0] from a rice bran extract; and
(b) promoting IL-6 production in dendritic cells of a human by administering an effective amount of the isolated Cer[t18:0/22:0] to the human via oral administration or parenteral administration.
